(19) United States
(12) Patent Application Publication
(10) Pub. No.: US 2025/0302965 A1
(43) Pub. Date: Oct. 2, 2025

(54) Preparations of Basic pH Hydrotropes & Methods for Making Same

(71) Applicant: Aurora Pharmaceutical, Inc., Northfield, MN (US)

(72) Inventors: Richard Mihalik, Faribault, MN (US); Jared Olson, Lakeville, MN (US); Elicia Olson, South St. Paul, MN (US); Michael Strobel, Northfield, MN (US)

(21) Appl. No.: 18/974,982

(22) Filed: Dec. 10, 2024

Related U.S. Application Data
(60) Provisional application No. 63/608,378, filed on Dec. 11, 2023.

(51) Int. Cl. A61K 47/22; A61K 47/26

(52) U.S. Cl. CPC A61K 47/22; A61K 47/26

(57) ABSTRACT

An illustrative embodiment of basic pH hydrotrope pharmaceutical preparation includes a hydrophobic polyaromatic organic compound (which polyaromatic organic compound may contain nitrogen) mixed with meglumine, wherein the meglumine is present in at least a 2:1 molar ratio of meglumine to polyaromatic compound, and wherein the hydrophobic polyaromatic compound contains at least two aromatic groups connected by a chain containing either a single secondary amine, a single amide, a single pyrazole, a single amino sulfone, or a single amino sulfone group attached to a single amide group. In other illustrative embodiments other additives may be included to increase the functionality of a preparation for a specific application.

### CROSS-REFERENCE TO RELATED APPLICATIONS

The present utility Non-provisional Patent application claims priority to provisional U.S. Patent Application No. 63/608,378, filed on Dec. 11, 2023, which application is incorporated by reference herein in its entirety.

### FIELD OF THE INVENTION

The present disclosure relates to the formation of basic pH hydrotropes having increased solubilization of certain hydrophobic and/or water-insoluble polyaromatic organic compounds. In one illustrative embodiment the polyaromatic organic compound contains nitrogen and that compound is mixed with meglumine, where the meglumine is present in at least a 2:1 molar ratio.

### BACKGROUND

Meglumine is a sugar alcohol with a terminal amino group substituted for an alcohol having the chemical structure shown below and the chemical formula C₇H₁₇NO₅. The molecular weight is 195.216 g/mol and the acidity (pKₐ) is from 8.0 to 9.6.

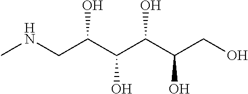

Meglumine has historically been used to form soluble salts with organic compounds often containing aryl halides such as the NSAID salt flunixin meglumine. Meglumine has also been used to form the iodinated contrast medias diatrizoate meglumine, iothalamate meglumine, and iodipamide meglumine in solutions. More recently, it has been used to form salts at lower pH values to make more water-soluble powders for pharmaceutical use and to increase solubility of aromatic compounds for polyaromatic synthesis. None of this earlier work identified basic pH values as important to the application or considered meglumine concentrations higher than a 1:1 molar ratio important to the solubilization, as the intent of the work was to form a soluble salt, not a hydrotrope in the form of a higher order chelate.

Meloxicam is an important NSAID in both human and animal health that is used both parenterally and orally that is practically insoluble in water. Human oral preparations are typically suspensions, gelatin capsules, and tablets. Animal oral preparations are typically oral suspensions. This is due to the limited water solubility of meloxicam. There is also a human parenteral preparation that is a non-aqueous solution in combination with bupivacaine that contains no meglumine. There are also aqueous parenteral preparations for animals that contain approximately 0.5% meloxicam, approximately 10% alcohol as a cosolvent, glycofurol as a cosolvent, Poloxamer 188 (available commercially as a non-ionic surfactant) as a cosolvent, sodium chloride, glycine, and 0.3% meglumine. The pH of these formulations is typically acidic at around pH 3.5-4.5.

In contrast, the meglumine in an illustrative embodiment of a formula according to the present disclosure is surprising given a pKₐ of 8.01 for meglumine. At a pH of 3.5-4.5, it is not acting as a substantive buffer, and it is suspected it was being used in the prior art along with the alcohol, glycofurol, Poloxamer 188, and glycine to help solubilize the meloxicam as a salt of meglumine. At 0.5% meloxicam and 0.3% meglumine (by weight/volume), the molar ratio is 1:1, which suggests that the formulator was trying to make a salt of the meloxicam in a 1:1 molar ratio. The use of Poloxamer 188 in the solution further suggests the formulator was trying to stabilize the salt in solution by forming a micelle around the 1:1 salt complex.

Known prior art that may be relevant to the present disclosure includes the following references but is not limiting to the scope of the present disclosure unless otherwise indicated in the following claims.

US Pat. Pub. 2014/0113893 A1 describes a formula for needleless injection of meglumine with meloxicam, but specifies it forms a salt with meloxicam. It is clear from the disclosure that the invention described in this reference requires other elements to solubilize the meloxicam/Meglumine salt. Also, the example provided requires heating to 90° C. to initially form the salt. Claim four requires meglumine and meloxicam are present at a molar ratio of 9:8 to 12:8 and claim five recites a molar ratio of 10:8. There is nothing in the examples suggesting solubility enhancement at higher ratios without the other components in the invention.

ES 2374626T3 teaches solubilizing the salt at the 1:1 molar basis and claim 1 requires either DMA or propylene glycol.

US Pat. Pub. 2021/0106591 A1 details the use of meglumine for buffering or pH adjustment.

U.S. Pat. No. 4,748,174A discloses that “the water soluble salts according to this reference are prepared by direct reaction between meglumine or glucamine with the NSAIDs R1 in equimolecular amounts.”

Labeling for Metacam® Injection indicates that Meglumine and meloxicam are present at a 1:1 ratio, which indicates that this formulation appears to be making the same salt as discussed above in other prior art references.

As such, an unmet need exists for meglumine concentrations higher than a 1:1 molar ratio in order to prepare a basic pH hydrotope of certain types of hydrophobic and/or water-insoluble polyaromatic organic compounds, which polyaromatic organic compounds may contain nitrogen. However, the scope of the present disclosure is not so limited and other unmet needs and/or advantageous features of the present disclosure may exist without departing from the scope of the present disclosure unless otherwise indicated in the following claims.

### SUMMARY

In accordance with one illustrative embodiment of a composition and/or preparation according to the present disclosure, a meloxicam formula was developed in a basic solution at a pH of 9.6 with meglumine at a much higher molar ratio of about 2:1 (meglumine to polyaromatic compound). In one illustrative embodiment, a chelated polyaromatic meloxicam formula was prepared. In another embodiment, the solution was buffered using meglumine at a more physically acceptable pH than the arginine chelate made in the prior art. Other illustrative embodiments and/or aspects of illustrative embodiments are disclosed herein.

### DETAILED DESCRIPTION

Before the present methods and composition, hydrotropes, and/or preparations are disclosed and described, it is to be understood that the methods and composition, hydrotropes, and/or preparations are not limited to specific methods, specific components, or to particular implementations unless so indicated in the following claims. It is also to be understood that the terminology used herein is for the purpose of describing particular illustrative embodiments/aspects only and is not intended to be limiting. Additionally, the terms “hydrotrope,” “preparation,” “chelate,” and “chelate system” may be used interchangeably where technically appropriate to describe various features or characteristics of illustrative embodiments of the present disclosure and those terms are not meant to be limited to a specific modality unless otherwise indicated in the following claims.

As used in the specification and the appended claims, the singular forms “a,” “an,” and “the” include plural referents unless the context clearly dictates otherwise. Ranges may be expressed herein as from “about” one particular value and/or to “about” another particular value. Unless otherwise stated, ranges herein include their endpoints. In some embodiments, and unless otherwise indicated, terms presented in singular form also include the plural situation. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Aspect” when referring to a method, composition, hydrotrope, and/or preparation, and/or component thereof does not mean that limitation, functionality, component, etc. referred to as an aspect is required, but rather that it is one part of a particular illustrative disclosure and not limiting to the scope of the method, composition, hydrotrope, and/or preparation, and/or component thereof unless so indicated in the following claims.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and make and use the disclosed compositions, hydrotropes, and/or preparations. These and other components are disclosed herein, and it is understood that when combinations, subsets, interactions, groups, etc. of these components are disclosed that while specific reference of each various individual and collective combinations and permutations of these may not be explicitly disclosed, each is specifically contemplated and described herein, for all methods and compositions, hydrotropes, and/or preparations. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and compositions and/or preparations may be understood more readily by reference to the following detailed description of preferred and illustrative aspects and embodiments, and the examples included therein. Corresponding terms may be used interchangeably when referring to generalities of configuration and/or corresponding components, aspects, features, functionality, methods and/or materials of construction, etc.

In order to try to solubilize meloxicam, a chelate with L-arginine was successfully made. Chelation with arginine requires a relatively high pH, so a chelation agent was sought that could achieve the same result at a somewhat lower pH. A meloxicam formula was successfully developed in a basic aqueous solution at a pH of 9.6 with meglumine at a much higher molar ratio than was recognized in the prior art in an attempt to both chelate the polyaromatic meloxicam and to buffer the solution using meglumine at a more physiologically acceptable pH than the arginine chelate made previously. Meglumine was chosen for its lower pKₐ relative to L-arginine and its ability to act as a buffering agent.

The initial work was developed and successfully formulated with the following formula:
1.50% w/v meloxicam
9.00% w/v meglumine (5:1 meglumine to meloxicam on a molar basis)
10.0% 190 proof ethanol
10.0% glycerin
0.20% xanthan gum
pH adjust Hydrochloric Acid
QS water
Resulting pH: 9.63

Two additional formulations were made at 0.05% w/v meloxicam (lab lot RDJO110922A) and 0.15% w/v meloxicam (lab lot RDJO110922B) with 0.9% meglumine each (molar ratios of 150:1 and 50:1, respectively). Assay stability at the same temperatures was also demonstrated for the 0.05% formula for 6 months, indicating that the formulation was very stable from at least 0.05% to 1.5% w/v meloxicam.

To determine whether the solution was stable because the meglumine was chelated, or because it was buffered, or for some combination of both, and to determine whether the meglumine was chelated by only one meglumine molecule or multiple meglumine molecules, a series of additional formulations were made. These additional formulations were made at molar ratios (meglumine to meloxicam) of 5:1, 3:1, 2:1, and 1:1 (lab lots RDJO020323, -323A, -323B, and -323C). All were soluble except the 1:1 ratio, indicating that the chelation to solubilize requires a 2:1 ratio, and any excess simply serves as buffer. It also surprisingly teaches against the prior art involving salts of meglumine where only a 1:1 ratio was required, but without limitation unless otherwise indicated in the following claims. The failure to solubilize at the 1:1 ratio suggested a different mechanism of solubilization from the prior art. To confirm that the formula was only stable at basic pH values, a sample of the 0.150% w/v meloxicam formula containing a 2:1 meglumine-to-meloxicam ratio on a molar basis was neutralized and fell out of solution when the pH crossed the neutral pH.

For additional empirical information, syrup formulations without ethanol were also formulated with a 2:1 meglumine-to-meloxicam ratio containing no ethanol, which demonstrate that the solubilization is not dependent on the ethanol and suggests that ethanol is instead primarily or only acting as a preservative, but without limitation unless otherwise indicated in the following claims.

From these results it was clear that a new method of making a hydrotrope and/or preparation of meloxicam had been identified. This spurred further empirical analysis with other pharmaceutical molecules to try to identify the functional groups or combination of structural groups necessary to form this newly identified type of chelating hydrotrope and/or preparation. This work elucidated that other hydrophobic and/or water-insoluble polyaromatic molecules in addition to meloxicam could be chelated at a basic pH with meglumine at a 2:1 molar ratio (meglumine to polyaromatic molecule) and eventually to the identification of the scope and combination of structural elements within the molecule necessary to form this newly identified type of hydrotrope and/or preparation.

As the investigation progressed, it was determined that meglumine at a basic pH was a very complicated chelating agent in that it is surprisingly only suitable to dissolve a very limited scope of molecules under a limited set of conditions. This led to many unsuccessful attempts to chelate molecules and several successful attempts. This experimentation led to the identification of necessary structural elements required for solubilization under basic conditions with meglumine at a 2:1 molar ratio (meglumine to polyaromatic molecule).

Analyzing the structural similarities and differences of the successful and unsuccessful molecules, the structural scope of the illustrative embodiments of compositions, hydrotropes, and/or preparations according to the present disclosure was identified. To be effective in this illustrative embodiment of a chelate system, preparation, hydrotrope, and/or or method, the molecules contain at least two aromatic groups that are connected by a chain or chains containing either a single secondary amine, a single amide, a single pyrazole, a single amino sulfone, or a single amino sulfone attached to a single amide group without limitation unless otherwise indicated in the following claims. A summary of certain water-insoluble molecules that were then solubilized by employing this illustrative embodiment of a chelate system, preparation, hydrotrope, and/or or method described herein is presented in Table 1 below. These examples are for illustrative purposes only and not meant to limit the scope of the present disclosure unless otherwise indicated in the following claims. Other water-insoluble molecules may become soluble therein by utilizing an illustrative embodiment of a chelate system, preparation, hydrotrope, and/or method disclosed herein without limitation unless otherwise indicated in the following claims.

TABLE 1
Summary of the water-insoluble molecules solubilized by employing and illustrative embodiment of the chelation mechanism,
preparation, hydrotrope, and/or or method described herein.

| | Reported | | Examples of | |
|---|---|---|---|---|
| | Solubility | | Solubilities | |
| | in | | Demonstrated | |
| | Water | | in Experiments | |
| Molecule | (in w/v) | Chemical Structure | (in w/v) | Comments |
| Meloxicam | Practically insoluble (Merck Index) | 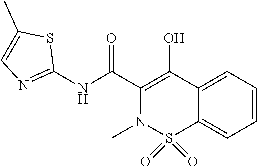 [SMILES: Cc1cnc(NC(=O)C2=C(O)c3ccccc3S(=O)(=O)N2C)s1] | At least 1.5% | Contains two aromatic groups separated by an amide |
| Nitazoxanide | 0.000755% (Pubchem) | 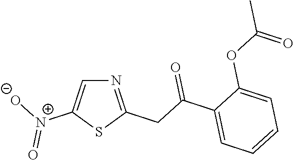 [SMILES: CC(=O)Oc1ccccc1C(=O)Cc1ncc([N+](=O)[O-])s1] | At least 1.0% | Contains two aromatic groups separated by an amide. |
| Piroxicam | 0.00497% (Pubchem) | 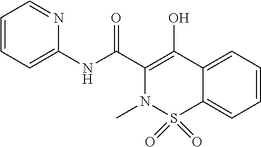 [SMILES: CN1C(C(=O)Nc2ccccn2)=C(O)c2ccccc2S1(=O)=O] | At least 0.15% | Contains two aromatic groups separated by a chain containing both an amide and an amino sulfone |
| Grapiprant | Insoluble (Pubchem) | 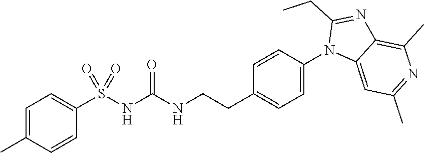 | At least 1.5% | Contains two aromatic groups separated by a chain containing an amino sulfone and an amide |
| Sulfadiazine | 0.0077% (Pubchem) | 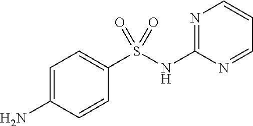 [SMILES: Nc1ccc(S(=O)(=O)Nc2ncccn2)cc1] | At least 30% | Contains two aromatic groups separated by an amino sulfone |
| Sulfadimethoxine | 0.0343% (Pubchem) | 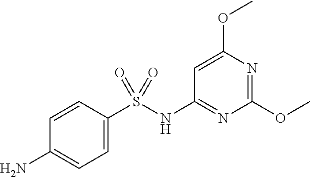 [SMILES: COc1cc(NS(=O)(=O)c2ccc(N)cc2)nc(OC)n1] | At least 12.5% | Contains two aromatic groups separated by an amino sulfone |
| Robenacoxib | 0.017% (published literature) | 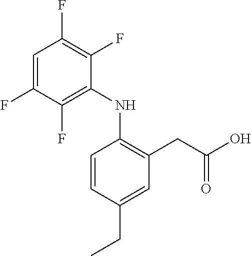 [SMILES: CCc1ccc(Nc2c(F)c(F)cc(F)c2F)c(CC(=O)O)c1] | At least 1.0% | Contains two aromatic groups separated by a secondary amine |
| Carprofen | 0.00411% (Pubchem) | 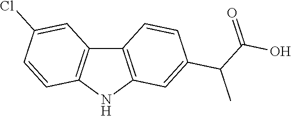 [SMILES: CC(C(=O)O)c1ccc2c(c1)[nH]c1ccc(Cl)cc12] | At least 10% | Contains two aromatic groups separated by a secondary amine |
| Phenylbutazone | 0.00463% (Pubchem) | 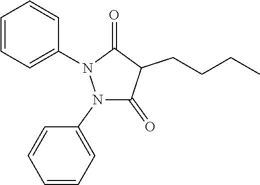 [SMILES: CCCCC1C(=O)N(c2ccccc2)N(c2ccccc2)C1=O] | At least 45% | Contains two aromatic groups separated by two amides |

Additional components may be added alone or in combination with one another to a solubilized system containing a previously water-insoluble polyaromatic organic compound to provide additional benefits for specific applications. For example, in one illustrative embodiment a preparation and/or hydrotrope containing a hydrophobic polyaromatic organic compound (which polyaromatic organic compound may contain nitrogen) mixed with meglumine may further include a wetting agent. Any suitable wetting agent for the particular application may be used, including but not limited to polysorbate, polyethylene glycol, sucrose esters, lauryl betaine, disodium laureth sulfosuccinate, sodium lauryl sulfate, sodium stearate, and/or combinations thereof without limitation unless otherwise indicated in the following claims.

In another illustrative embodiment a preparation and/or hydrotrope containing a hydrophobic polyaromatic organic compound (which polyaromatic organic compound may contain nitrogen) mixed with meglumine may further include a preservative. Any suitable preservative for the particular application may be used, including but not limited to ethanol, isopropyl alcohol, butyl alcohol, propylene glycol, phenoxyethanol, benzyl alcohol, caprylyl glycol, sodium benzoate, potassium sorbate, chlorphenesin, sodium dehydroacetate, tetrasodium EDTA, tea tree oil, rosemary extract, thyme oil, and/or combinations thereof without limitation unless otherwise indicated in the following claims.

In another illustrative embodiment a preparation and/or hydrotrope containing a hydrophobic polyaromatic organic compound (which polyaromatic organic compound may contain nitrogen) mixed with meglumine may further include an antioxidant. Any suitable antioxidant for the particular application may be used, including but not limited to butyl hydroxy toluene, tertiary butylhydroquinone, propyl gallate, gallic acid, sodium ascorbate, vitamin E, vitamin C, resveratrol, green tea extract, coenzyme Q10, alpha-lipoic acid, rosemary extract, sage extract, oregano extract, milk thistle, and/or combinations thereof without limitation unless otherwise indicated in the following claims.

In another illustrative embodiment a preparation and/or hydrotrope containing a hydrophobic polyaromatic organic compound (which polyaromatic organic compound may contain nitrogen) mixed with meglumine may further include a flavoring compound and/or palatability enhancer. Any suitable flavoring compound and/or palatability enhancer for the particular application may be used, including but not limited to strawberry flavoring, raspberry flavor (ethyl 2-methylbutyrate, raspberry ketone), cherry flavor (benzaldehyde, ethyl butyrate), peach flavor (ethyl acetate, gamma-undecalactone), citrus (limonene, linalool, citral), apple flavor (ethyl 2-methylbutyrate, hexyl acetate), pineapple flavor (ethyl 2-methylbutyrate, pineapple lactone), grape flavor (isoamyl acetate, methyl anthranilate), blackberry flavor (furfural, methyl octanoate), blueberry flavor (methyl 2-methylbutyrate, linalool), cranberry flavor (cranberry lactone, methyl salicylate), acai berry flavor (acai oil, acai berry extract), and/or combinations thereof without limitation unless otherwise indicated in the following claims.

In another illustrative embodiment a preparation and/or hydrotrope containing a hydrophobic polyaromatic organic compound (which polyaromatic organic compound may contain nitrogen) mixed with meglumine may further include a suspending agent. Any suitable suspending agent for the particular application may be used, including but not limited to sodium carboxymethylcellulose, xanthan gum, guar gum, hydroxyethylcellulose, carboxymethyl starch, methylcellulose, pectin, polysorbate, sodium alginate, and/or combinations thereof without limitation unless otherwise indicated in the following claims.

In another illustrative embodiment a preparation and/or hydrotrope containing a hydrophobic polyaromatic organic compound (which polyaromatic organic compound may contain nitrogen) mixed with meglumine may further include a viscosity-increasing agent. Any suitable viscosity-increasing agent for the particular application may be used, including but not limited to xanthan gum, guar gum, hydroxyethylcellulose, carboxymethyl starch, methylcellulose, agar-agar, pectin, locust bean gum, tara gum, sodium alginate, xyloglucan, konjac gum, alginic acid, carrageenan, tapioca starch, modified starches, silica, polyacrylamide, and/or combinations thereof without limitation unless otherwise indicated in the following claims.

In another illustrative embodiment a preparation and/or hydrotrope containing a hydrophobic polyaromatic organic compound (which polyaromatic organic compound may contain nitrogen) mixed with meglumine may further include a viscosity-reducing agent. Any suitable viscosity-reducing agent for the particular application may be used, including but not limited to various suitable alcohols (e.g., ethanol, isopropyl alcohol, methanol, propylene glycol, butanol, etc.), glycerin, acetone, dimethyl sulfoxide, toluene, xylene, cyclohexane, diethylene glycol, hexane, methyl ethyl ketone, limonene, ethyl acetate, benzene, turpentine, naphtha, and/or combinations thereof without limitation unless otherwise indicated in the following claims.

In another illustrative embodiment a preparation and/or hydrotrope containing a hydrophobic polyaromatic organic compound (which polyaromatic organic compound may contain nitrogen) mixed with meglumine may further include an additional pharmaceutical active ingredient (in either solution, suspension, or a combination thereof). Any suitable additional pharmaceutical active ingredient for the particular application may be used, including but not limited to ivermectin, moxidectin, milbemycin oxime, eprinomectin, doramectin, afoxolaner, esafoxolaner, tigolaner, fluralaner, lotilaner, sarolaner, fenbendazole, albendazole, pyrantel pamoate, enrofloxacin, ciprofloxacin, tulathromycin, gentamicin, tilmicosin, trimethoprim, pyrimethamine, amprolium, marbofloxacin, praziquantel, imidacloprid, febantel, epsiprantel, amoxicillin, spectinomycin, florfenicol, clindamycin tylosin, tyvalosin, clorsulon, levamisole, orbifloxacin, spinosad, lufenuron, danofloxacin, nitenpyram, ponazuril, diclazuril, and salts thereof, and/or combinations thereof without limitation unless otherwise indicated in the following claims.

All the various additives and/or additional pharmaceutical active ingredients disclosed herein may be used alone or in combination with one another depending on the suitability of same for a specific application without limitation unless otherwise indicated in the following claims. It is contemplated that the optimal additives and/or additional pharmaceutical active ingredients will vary from one application to the next and are therefore in no way limiting to the scope of the present disclosure unless otherwise indicated in the following claims.

The specific pH value of the resulting preparation and/or hydrotrope may be between 5 and 13 for various applications, and more preferably between 7 and 12 for other applications, more preferably between 8 and 10.5 for other applications, and even more preferably between 9 and 10 for other applications without limitation unless otherwise indicated in the following claims. Any suitable buffer and/or pH-adjusting agent may be used for a specific application, and the optimal buffer and/or pH-adjusting agent may vary on the specific application without limitation unless otherwise indicated in the following claims. The concentration of the hydrophobic polyaromatic organic compound within the preparation may vary from one illustrative embodiment to another, and the optimal concentration thereof may vary from one application to another and is therefore in no way limiting to the scope of the present disclosure unless otherwise indicated in the following claims. It is contemplated that suitable concentrations for certain applications may include but are not limited to from 0.01% to 45% weight per volume, and more preferably 0.025% to 40% weight per volume, and more preferably still 0.05% to 33.3% weight per volume.

Non-limiting embodiments of a hydrodrope, preparation, and/or composition include:

1. A basic pH hydrotrope pharmaceutical preparation of a hydrophobic polyaromatic compound mixed with meglumine, wherein the meglumine is present in at least a 2:1 molar ratio of meglumine to polyaromatic compound, and wherein the hydrophobic polyaromatic compound contains at least two aromatic groups connected by a chain containing either a single secondary amine, a single amide, a single pyrazole, a single amino sulfone, or a single amino sulfone group attached to a single amide group.

2. The preparation of claim 1 wherein the hydrophobic polyaromatic organic compound is further defined as an organic compound containing nitrogen.

3. A hydrotrope of claim 1 where the hydrophobic polyaromatic compound is further defined as meloxicam, nitazoxanide, piroxicam, grapiprant, sulfadiazine, sulfadimethoxine, carprofen, robenacoxib, tenoxicam, droxicam, phenylbutazone, diclofenac, mefenamic acid, sulfapyridine, and combinations thereof.

4. The preparation of claim 1 further comprising a wetting agent.

5. The preparation of claim 1 further comprising a preservative.

6. The preparation of claim 1 further comprising an antioxidant.

7. The preparation of claim 1 further comprising a palatability enhancer.

8. The preparation of claim 1 further comprising a suspending agent.

9. The preparation of claim 1 further comprising a viscosity-increasing agent.

10. The preparation of claim 1 further comprising a viscosity-reducing agent.

11. The preparation of claim 1 further comprising a pharmaceutical active ingredient in suspension or solution.

12. The preparation of claim 11 wherein the pharmaceutical active ingredient is further defined as ivermectin, moxidectin, milbemycin oxime, eprinomectin, doramectin, afoxolaner, esafoxolaner, tigolaner, fluralaner, lotilaner, sarolaner, fenbendazole, albendazole, pyrantel pamoate, enrofloxacin, ciprofloxacin, tulathromycin, gentamicin, tilmicosin, trimethoprim, pyrimethamine, amprolium, marbofloxacin, praziquantel, imidacloprid, febantel, epsiprantel, amoxicillin, spectinomycin, florfenicol, clindamycin tylosin, tyvalosin, clorsulon, levamisole, orbifloxacin, spinosad, lufenuron, danofloxacin, nitenpyram, ponazuril, diclazuril, and salts thereof, and combinations thereof.

13. The preparation of claim 1 wherein the meglumine is present preferably at a minimum of about a 2:1 molecular ratio of meglumine to hydrophobic polyaromatic compound up to the limit of solubility of meglumine in the formulation, wherein an excess of meglumine beyond the 2:1 ratio acts as a buffer.

14. The preparation of claim 1 wherein the meglumine is present preferably at about a 2:1 to 5:1 molecular ratio of meglumine to hydrophobic polyaromatic compound, and wherein an excess meglumine beyond the 2:1 ratio acts as a buffer.

15. The preparation of claim 1 wherein the meglumine is present at about a 2:1 molecular ratio of meglumine to hydrophobic polyaromatic compound if no additional buffering is desired from the meglumine.

16. The preparation of claim 1 wherein a pH of the preparation is between 7 and 12.

17. The preparation of claim 1 wherein the pH of the preparation is between 8 and 10.5.

18. The preparation of claim 1 wherein the pH of the preparation is between 9 and 10.

19. The preparation of claim 3 wherein the hydrophobic polyaromatic compound is present at about 0.01% to 45% weight per volume.

20. The preparation of claim 3 wherein the polyaromatic compound is present at about 0.025% to 40% weight per volume.

21. The preparation of claim 3 wherein the polyaromatic compound is at about 0.05% to 33.3% weight per volume.

22. The preparation of claim 4 wherein the wetting agent is selected from a group consisting of polysorbate, polyethylene glycol, sucrose esters, lauryl betaine, disodium laureth sulfosuccinate, sodium lauryl sulfate, sodium stearate, and combinations thereof.

23. The preparation of claim 5 wherein the preservative is selected from a group consisting of ethanol, isopropyl alcohol, butyl alcohol, propylene glycol, phenoxyethanol, benzyl alcohol, caprylyl glycol, sodium benzoate, potassium sorbate, chlorphenesin, sodium dehydroacetate, tetrasodium EDTA, tea tree oil, rosemary extract, thyme oil, and combinations thereof.

24. The preparation of claim 6 wherein the antioxidant is selected from a group consisting of butyl hydroxy toluene, tertiary butylhydroquinone, propyl gallate, gallic acid, sodium ascorbate, vitamin E, vitamin C, resveratrol, green tea extract, coenzyme Q10, alpha-lipoic acid, rosemary extract, sage extract, oregano extract, milk thistle, and combinations thereof.

25. The preparation of claim 7 wherein the flavoring compound is selected from a group consisting of strawberry flavoring, raspberry flavor (ethyl 2-methylbutyrate, raspberry ketone), cherry flavor (benzaldehyde, ethyl butyrate), peach flavor (ethyl acetate, gamma-undecalactone), citrus (limonene, linalool, citral), apple flavor (ethyl 2-methylbutyrate, hexyl acetate), pineapple flavor (ethyl 2-methylbutyrate, pineapple lactone), grape flavor (isoamyl acetate, methyl anthranilate), blackberry flavor (furfural, methyl octanoate), blueberry flavor (methyl 2-methylbutyrate, linalool), cranberry flavor (cranberry lactone, methyl salicylate), acai berry flavor (acai oil, acai berry extract), and combinations thereof.

26. The preparation of claim 8 wherein the suspending agent is selected from a group consisting of sodium carboxymethylcellulose, xanthan gum, guar gum, hydroxyethylcellulose, carboxymethyl starch, methylcellulose, pectin, polysorbate, sodium alginate, and combinations thereof.

27. The preparation of claim 9 wherein the viscosity-increasing agent is selected from a group consisting of xanthan gum, guar gum, hydroxyethylcellulose, carboxymethyl starch, methylcellulose, agar-agar, pectin, locust bean gum, tara gum, sodium alginate, xyloglucan, konjac gum, alginic acid, carrageenan, tapioca starch, modified starches, silica, polyacrylamide, and combinations thereof.

28. The preparation of claim 10 wherein the viscosity-reducing agent is selected from a group consisting of alcohols, glycerin, acetone, dimethyl sulfoxide, toluene, xylene, cyclohexane, diethylene glycol, hexane, methyl ethyl ketone, limonene, ethyl acetate, benzene, turpentine, naphtha, and combinations thereof.

29. The preparation of claim 11 wherein the pharmaceutical active ingredient is selected from a group consisting of ivermectin, moxidectin, milbemycin oxime, eprinomectin, doramectin, afoxolaner, esafoxolaner, tigolaner, fluralaner, lotilaner, sarolaner, fenbendazole, albendazole, pyrantel pamoate, enrofloxacin, ciprofloxacin, tulathromycin, gentamicin, tilmicosin, trimethoprim, pyrimethamine, amprolium, marbofloxacin, praziquantel, imidacloprid, febantel, epsiprantel, amoxicillin, spectinomycin, florfenicol, clindamycin tylosin, tyvalosin, clorsulon, levamisole, orbifloxacin, spinosad, lufenuron, danofloxacin, nitenpyram, ponazuril, diclazuril, and salts thereof, and combinations thereof.

30. A basic pH hydrotrope pharmaceutical preparation of a hydrophobic polyaromatic compound containing nitrogen mixed with meglumine, wherein the meglumine is present in at least a 2:1 molar ratio of meglumine to polyaromatic compound, and wherein the hydrophobic polyaromatic compound contains at least two aromatic groups connected by a chain containing either a single secondary amine, a single amide, a single pyrazole, a single amino sulfone, or a single amino sulfone group attached to a single amide group.

Although various illustrative embodiments of the present disclosure utilize hydrophobic polyaromatic organic compounds that contain nitrogen, the scope of the present disclosure is not so limited unless otherwise indicated in the following claims, and other illustrative embodiments may utilize polyaromatic organic compounds without nitrogen.

Through testing and analysis it has been determined that various other water-insoluble molecules remained insoluble after treatment to create a chelate system. A summary of these water-insoluble molecules and comments are provided below in Table 2. However, such water-insoluble molecules and/or solubility results regarding same are for illustrative purposes only and are in no way limiting to the scope of the present disclosure unless otherwise indicated in the following claims.

TABLE 2
Summary of the water-insoluble molecules that remained insoluble after treatment to create a chelate system.

| Molecule | Structure | Comments |
|---|---|---|
| Moxidectin | 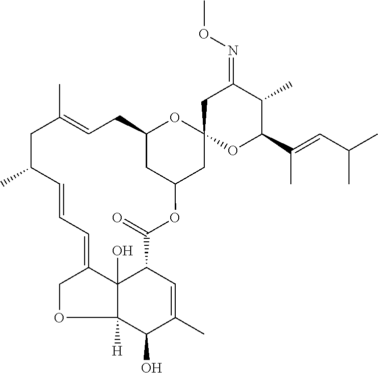 [SMILES: CO/N=C1\C[C@]2(CC3C[C@@H](C/C=C(\C)C[C@@H](C)/C=C/C=C4\CO[C@@H]5[C@H](O)C(C)=C[C@@H](C(=O)O3)C45O)O2)O[C@H](/C(C)=C/C(C)C)[C@H]1C] | No amines, amides, amino sulfones, or aromatic groups and did not dissolve when tested. |
| Eprinomectin | 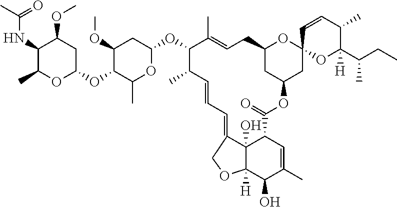 [SMILES: CC[C@H](C)[C@H]1O[C@]2(C=C[C@@H]1C)C[C@@H]1C[C@@H](C/C=C(\C)[C@@H](O[C@H]3C[C@H](OC)[C@@H](O[C@H]4C[C@H](OC)[C@H](NC(C)=O)[C@H](C)O4)C(C)O3)[C@@H](C)/C=C/C=C3\CO[C@@H]4[C@H](O)C(C)=C[C@@H](C(=O)O1)[C@]34O)O2] | Contains an amide, but no amines, amino sulfones or aromatic groups and did not dissolve when tested. |
| Florfenicol | 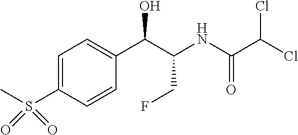 [SMILES: CS(=O)(=O)c1ccc([C@@H](O)[C@@H](CF)NC(=O)C(Cl)Cl)cc1] | Contains an amide and an aryl sulfone, but only one aromatic group and did not dissolve when tested. |
| Deracoxib | 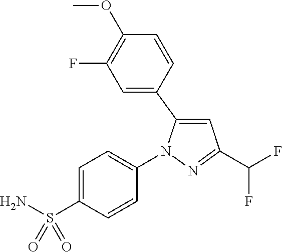 [SMILES: COc1ccc(-c2cc(C(F)F)nn2-c2ccc(S(N)(=O)=O)cc2)cc1F] | Contains two aromatic groups and an aryl amino sulfone but the two nitrogen atoms between the aromatic groups are not secondary amines, amides or amino sulfones and did not dissolve when tested. |
| Firocoxib | 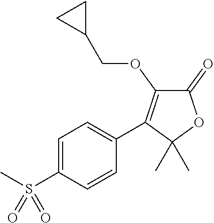 [SMILES: CC1(C)OC(=O)C(OCC2CC2)=C1c1ccc(S(C)(=O)=O)cc1] | Does not contain any nitrogen and only one aromatic group with an aryl sulfone and did not dissolve when tested. |
| Imidacloprid | 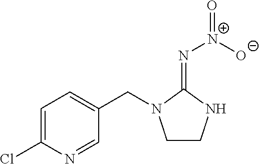 [SMILES: O=[N+]([O-])/N=C1\NCCN1Cc1ccc(Cl)nc1] | Contains a secondary amine and other nitrogen containing groups but only one aromatic group and did not dissolve when tested. |
| Lidocaine | 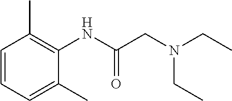 [SMILES: CCN(CC)CC(=O)Nc1c(C)cccc1C] | Contains an amide and a tertiary amine, but only one aromatic group and did not dissolve when tested. |
| Enrofloxacin | 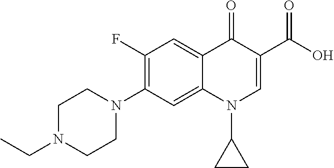 [SMILES: CCN1CCN(c2cc3c(cc2F)c(=O)c(C(=O)O)cn3C2CC2)CC1] | Contains only tertiary amines and only one aromatic group and did not dissolve when tested. |
| Sulfanilamide | 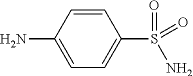 [SMILES: Nc1ccc(S(N)(=O)=O)cc1] | Contains a primary aryl amine and an amino sulfone, but only one aromatic group and did not dissolve when tested. |
| Afoxolaner | 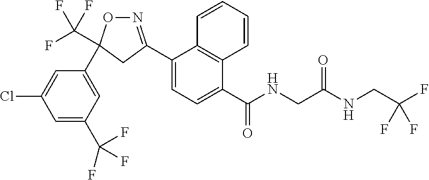 [SMILES: O=C(CNC(=O)c1ccc(C2=NOC(c3cc(Cl)cc(C(F)(F)F)c3)(C(F)(F)F)C2)c2ccccc12)NCC(F)(F)F] | Contains two amides and two aromatic groups, but the amides are not on a chain between the two aromatic groups. The one nitrogen atom that is between the two aromatic groups is not a secondary amine, amide or amino sulfone and did not dissolve when tested. |
| Trimethoprim | 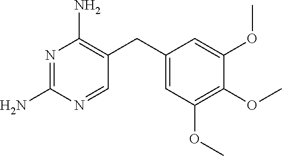 [SMILES: COc1cc(Cc2cnc(N)nc2N)cc(OC)c1OC] | Contains two primary aryl amines and two aromatic groups, but the primary amines are not on a chain between the two aromatic groups and did not dissolve when tested. |
| Fenbendazole | 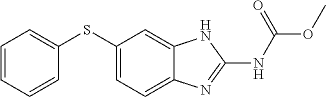 [SMILES: COC(=O)Nc1nc2ccc(Sc3ccccc3)cc2[nH]1] | Contains two aromatic groups and an amide, but the amide is not on the chain between the two aromatic groups and did not dissolve when tested. |
| Omeprazole | 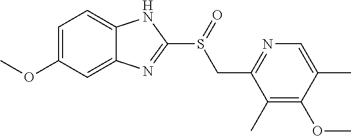 | Contains an aromatic benzene ring, a sulfoxide and an aromatic benzimidazole group, but it does not contain a secondary amine, amide or amino sulfone group and did not dissolve when tested. |
| Ponazuril | 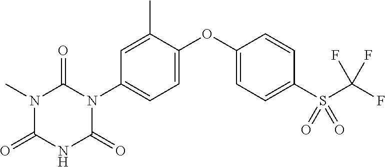 [SMILES: Cc1cc(-n2c(=O)[nH]c(=O)n(C)c2=O)ccc1Oc1ccc(S(=O)(=O)C(F)(F)F)cc1] | Contains two aromatic groups and a sulfoxide and an amide, but the amide is not on the chain between the two aromatic rings and did not dissolve when tested. |
| Lufenuron | 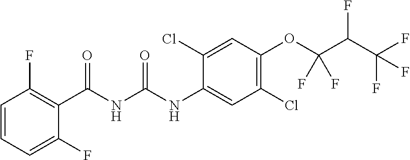 [SMILES: O=C(NC(=O)c1c(F)cccc1F)Nc1cc(Cl)c(OC(F)(F)C(F)C(F)(F)F)cc1Cl] | Contains two aromatic groups separated by two amide groups between the two aromatic groups and did not dissolve when tested. |

Meglumine is an amino derivative of a short chain sugar alcohol. To determine if the chelation would also be possible with other short chain sugar alcohols, the closely related glucosamine was tested at a 2:1 molar ratio in basic solution with meloxicam. It failed to solubilize the meloxicam under the same conditions. To confirm that this was not just an incompatibility with meloxicam, grapiprant and carprofen were also tried, and neither dissolved. This led to the conclusion that the illustrative embodiment utilizing meglumine may be most suitable for various applications, and certain applications may be suitable for utilizing other amino derivatives of short chain sugar alcohols without limitation unless otherwise indicated in the following claims.

Having described preferred aspects and embodiments of the various processes, compositions, preparations, and products made thereby, other features of the present disclosure will undoubtedly occur to those versed in the art, as will numerous modifications and alterations in the embodiments and/or aspects as illustrated herein, all of which may be achieved without departing from the spirit and scope of the present disclosure. Accordingly, the methods and embodiments pictured and described herein are for illustrative purposes only, and the scope of the present disclosure extends to all processes, compositions, preparations, and/or structures for providing the various benefits and/or features of the present disclosure unless so indicated in the following claims.

While the chemical process, process steps, compositions, preparations, components thereof, and products made thereby according to the present disclosure have been described in connection with preferred aspects and specific examples, it is not intended that the scope be limited to the particular embodiments and/or aspects set forth, as the embodiments and/or aspects herein are intended in all respects to be illustrative rather than restrictive unless otherwise indicated in the following claims. Accordingly, the processes and embodiments pictured and described herein are no way limiting to the scope of the present disclosure unless so stated in the following claims.

Although several figures are drawn to accurate scale, any dimensions provided herein are for illustrative purposes only and in no way limit the scope of the present disclosure unless so indicated in the following claims. It should be noted that the processes, compositions, preparations, water-insoluble molecules, and/or other molecules/compounds used therewith are not limited to the specific embodiments pictured and described herein, but rather the scope of the inventive features according to the present disclosure is defined by the claims herein. Modifications and alterations from the described embodiments will occur to those skilled in the art without departure from the spirit and scope of the present disclosure.

Any of the various features, components, functionalities, advantages, aspects, configurations, process steps, process parameters, etc. of a chemical process, a process step, a substrate, composition, and/or preparation, may be used alone or in combination with one another depending on the compatibility of the features, components, functionalities, advantages, aspects, configurations, process steps, process parameters, etc. Accordingly, a nearly infinite number of variations of the present disclosure exist. Modifications and/or substitutions of one feature, component, functionality, aspect, configuration, process step, process parameter, etc. for another in no way limit the scope of the present disclosure unless so indicated in the following claims.

It is understood that the present disclosure extends to all alternative combinations of one or more of the individual features mentioned, evident from the text and/or drawings, and/or inherently disclosed. All of these different combinations constitute various alternative aspects of the present disclosure and/or components thereof. The embodiments described herein explain the best modes known for practicing the compositions, preparations, methods, and/or components disclosed herein and will enable others skilled in the art to utilize the same. The claims are to be construed to include alternative embodiments to the extent permitted by the prior art.

Unless otherwise expressly stated in the claims, it is in no way intended that any process or method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including but not limited to: matters of logic with respect to arrangement of steps or operational flow; plain meaning derived from grammatical organization or punctuation; the number or type of embodiments described in the specification.

What is claimed is:

1. A basic pH hydrotrope pharmaceutical preparation of a hydrophobic polyaromatic compound mixed with meglumine, wherein the meglumine is present in at least a 2:1 molar ratio of meglumine to polyaromatic compound, and wherein the hydrophobic polyaromatic compound contains at least two aromatic groups connected by a chain containing either a single secondary amine, a single amide, a single pyrazole, a single amino sulfone, or a single amino sulfone group attached to a single amide group.

2. The preparation of claim 1 wherein the hydrophobic polyaromatic organic compound is further defined as an organic compound containing nitrogen.

3. A hydrotrope of claim 1 where the hydrophobic polyaromatic compound is further defined as meloxicam, nitazoxanide, piroxicam, grapiprant, sulfadiazine, sulfadimethoxine, carprofen, robenacoxib, tenoxicam, droxicam, phenylbutazone, diclofenac, mefenamic acid, sulfapyridine, and combinations thereof.

4. The preparation of claim 1 further comprising a wetting agent.

5. The preparation of claim 1 further comprising a preservative.

6. The preparation of claim 1 further comprising an antioxidant.

7. The preparation of claim 1 further comprising a palatability enhancer.

8. The preparation of claim 1 further comprising a suspending agent.

9. The preparation of claim 1 further comprising a viscosity-increasing agent.

10. The preparation of claim 1 further comprising a viscosity-reducing agent.

11. The preparation of claim 1 further comprising a pharmaceutical active ingredient in suspension or solution.

12. The preparation of claim 11 wherein the pharmaceutical active ingredient is further defined as ivermectin, moxidectin, milbemycin oxime, eprinomectin, doramectin, afoxolaner, esafoxolaner, tigolaner, fluralaner, lotilaner, sarolaner, fenbendazole, albendazole, pyrantel pamoate, enrofloxacin, ciprofloxacin, tulathromycin, gentamicin, tilmicosin, trimethoprim, pyrimethamine, amprolium, marbofloxacin, praziquantel, imidacloprid, febantel, epsiprantel, amoxicillin, spectinomycin, florfenicol, clindamycin tylosin, tyvalosin, clorsulon, levamisole, orbifloxacin, spinosad, lufenuron, danofloxacin, nitenpyram, ponazuril, diclazuril, and salts thereof, and combinations thereof.

13. The preparation of claim 1 wherein the meglumine is present preferably at a minimum of about a 2:1 molecular ratio of meglumine to hydrophobic polyaromatic compound up to the limit of solubility of meglumine in the formulation, wherein an excess of meglumine beyond the 2:1 ratio acts as a buffer.

14. The preparation of claim 1 wherein the meglumine is present preferably at about a 2:1 to 5:1 molecular ratio of meglumine to hydrophobic polyaromatic compound, and wherein an excess meglumine beyond the 2:1 ratio acts as a buffer.

15. The preparation of claim 1 wherein the meglumine is present at about a 2:1 molecular ratio of meglumine to hydrophobic polyaromatic compound if no additional buffering is desired from the meglumine.

16. The preparation of claim 1 wherein a pH of the preparation is between 7 and 12.

17. The preparation of claim 1 wherein the pH of the preparation is between 8 and 10.5.

18. The preparation of claim 1 wherein the pH of the preparation is between 9 and 10.

19. The preparation of claim 3 wherein the hydrophobic polyaromatic compound is present at about 0.01% to 45% weight per volume.

20. The preparation of claim 3 wherein the polyaromatic compound is present at about 0.025% to 40% weight per volume.

21. The preparation of claim 3 wherein the polyaromatic compound is at about 0.05% to 33.3% weight per volume.
